(19) United States
(12) Patent Application Publication
(10) Pub. No.: US 2010/0113481 A1
(43) Pub. Date: May 6, 2010

(54) USE OF SERUM AMYLOID A GENE IN DIAGNOSIS AND TREATMENT OF GLAUCOMA AND IDENTIFICATION OF ANTI-GLAUCOMA AGENTS

(75) Inventor: Wan-Heng Wang, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/638,072

(22) Filed: Dec. 15, 2009

Related U.S. Application Data
(63) Continuation-in-part of application No. 11/615,454, filed on Dec. 22, 2006, now Pat. No. 7,662,389.
Application No. 11/615,454 is a continuation-in-part of application No. 11/000,757, filed on Dec. 1, 2004, now Pat. No. 7,357,931.
(60) Provisional application No. 60/530,430, filed on Dec. 17, 2003.

(51) Int. Cl. A61K 31/506; A61P 27/02; A61P 27/06

(52) U.S. Cl. 514/256

(57) ABSTRACT

The present invention provides compositions and methods for treating glaucoma, methods for diagnosing glaucoma, and methods for identifying agents which may be useful in the treatment of glaucoma. More specifically, the present invention describes the use of agents that modulate the expression of serum amyloid A, wherein such agents are inhibitors of p38 MAPkinase.

The present invention is a continuation-in-part of U.S. application Ser. No. 11/615,454, filed Dec. 22, 2006 (now allowed), which is a continuation-in-part of U.S. application Ser. No. 11/000,757, filed Dec. 1, 2004 (now U.S. Pat. No. 7,357,931), which claims priority to U.S. provisional application No. 60/530,430, filed Dec. 17, 2003.

### BACKGROUND OF THE INVENTION

1. Field of the Invention

The present invention relates to the field of diagnosis and treatment of glaucoma. More specifically, the invention provides methods and compositions for diagnosing and treating glaucoma and for identifying agents potentially useful for the treatment of glaucoma.

2. Description of the Related Art

There are a number of ocular conditions that are caused by, or aggravated by, damage to the optic nerve head, degeneration of ocular tissues, and/or elevated intraocular pressure. For example, “glaucomas” are a group of debilitating eye diseases that are a leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Twenty years ago, the interplay of ocular hypertension, ischemia and mechanical distortion of the optic nerve head were heavily debated as the major factors causing progression of visual field loss in glaucoma. Since then, other factors including excitotoxicity, nitric oxide, absence of vital neurotrophic factors, abnormal glial/neuronal interplay and genetics have been implicated in the degenerative disease process. The consideration of molecular genetics deserves some discussion insofar as it may ultimately define the mechanism of cell death, and provide for discrimination of the various forms of glaucoma. Within the past 10 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congenital glaucoma (CYP1B1), two mapped genes for pigmentary dispersion/pigmentary glaucoma, and a number of genes for developmental or syndromic forms of glaucoma (FOXC1, PITX2, LMX1B, PAX6).

Thus, each form of glaucoma may have a unique pathology and accordingly a different therapeutic approach to the management of the disease may be required. For example, a drug that effects the expression of enzymes that degrade the extracellular matrix of the optic nerve head would not likely prevent RGC death caused by excitotoxicity. In glaucoma, RGC death occurs by a process called apoptosis (programmed cell death). It has been speculated that different types of insults that can cause death may do so by converging on a few common pathways. Targeting downstream at a common pathway is a strategy that may broaden the utility of a drug and increase the probability that it may have utility in the management of different forms of the disease. However, drugs that effect multiple metabolic pathways are more likely to produce undesirable side-effects. With the advent of gene-based diagnostic kits to identify specific forms of glaucoma, selective neuroprotective agents can be tested with the aim of reducing the degree of variation about the measured response.

Glaucoma is currently diagnosed based on specific signs of the disease (characteristic optic nerve head changes and visual field loss). However, over half of the population with glaucoma are unaware they have this blinding disease and by the time they are diagnosed, they already have irreversibly lost approximately 30-50% of their retinal ganglion cells. Thus, improved methods for early diagnosis of glaucoma are needed.

Current glaucoma therapy is directed to lowering TOP, a major risk factor for the development and progression of glaucoma. However, none of the current IOP lowering therapies actually intervenes in the glaucomatous disease process responsible for elevated TOP and progressive damage to the anterior segment continues. This is one possible reason why most patients become “resistant” to conventional glaucoma therapies. Thus, what is needed is a therapeutic method for altering (by inhibiting or even reversing) the disease process.

### SUMMARY OF THE INVENTION

The present invention overcomes these and other drawbacks of the prior art by providing methods to diagnose and compositions to treat glaucoma. In one aspect, the present invention provides a method for treating glaucoma by administering to a patient in need thereof a therapeutically effective amount of a composition comprising an agent that interacts with a gene encoding serum amyloid A protein (SAA), or with the gene's promoter sequence. The interaction between the agent and the gene encoding SAA, or with its promoter sequence, modulates the expression of SAA, such that the patient's glaucomatous condition is treated. In preferred embodiments, the agent will be a protein, peptide, peptidomimetic, small molecule or nucleic acid.

In another aspect, the present invention provides a method for treating glaucoma by administering to a patient in need thereof a therapeutically effective amount of a composition comprising an agent that inhibits interaction of the serum amyloid A protein (SAA) with its receptor. Preferably, the agent will be a peroxisome proliferator-activated receptor α (PPARα) agonists, tachykinin peptides and their non-peptide analogs, p38MAPKinase inhibitors, or α-lipoic acid. Most preferably, the agent will be fenofibrate, Wy-14643, (4-chloro-6-(2,3-xylidino)-2-pryrimidinylthiol)-acetic acid), ciprofibrate, 2-bromohexadecanoic acid, bezafibrate, ciglitizone, bafilomycin, concanamycin, or pseudolaric acid B. In other aspects, the compound will be a p38MAPKinase inhibitor described in WO2005/105790, incorporated herein by reference. Preferred compounds for use in the methods of the invention include compounds described in Examples 1-136 and compounds I-1 through I-69 of WO2005/105790. More preferably, the compound for use in the methods of the invention will be the compounds described in Examples 10 and 14 of WO2005/105790.

The present invention further provides a pharmaceutical composition for treating glaucoma comprising a therapeutically effective amount of a serum amyloid A protein (SAA) antagonist and a pharmaceutical carrier. The antagonist contained in the composition may be any of the compounds identified above.

In yet another embodiment, the present invention provides a method for diagnosing glaucoma, by the following steps:
a) obtaining a biological sample from a patient; and
b) analyzing said sample for an aberrant level, aberrant bioactivity or mutations of the gene encoding serum amyloid A protein (SAA) or its promoter region or its gene products, wherein said gene encoding SAA comprises the sequence set forth in SEQ ID NO:1 or SEQ ID NO:3, wherein its promoter region comprises the sequence set forth in SEQ ID NO:12 or SEQ ID NO:13, and wherein SAA comprises the sequence set forth in SEQ ID NO:2 or SEQ ID NO:4;
wherein the aberrantly high level, aberrantly high bioactivity or mutations of the SAA genes or the gene products indicates a diagnosis of glaucoma.

In preferred aspects, the biological sample is ocular tissue, tears, aqueous humor, cerebrospinal fluid, nasal or cheek swab or serum. Most preferably, the biological sample comprises trabecular meshwork cells.

Alternatively, the present invention provides a method for diagnosing glaucoma in a patient, by the steps:
a) collecting cells from a patient;
b) isolating nucleic acid from the cells;
c) contacting the sample with one or more primers which specifically hybridize 5′ and 3′ to at least one allele of SEQ ID NO:1, SEQ ID NO:3, SEQ ID NO:12, or SEQ ID NO:13 under conditions such that hybridization and amplification of the allele occurs; and
d) detecting the amplification product;
wherein aberrant level or mutations of SEQ ID NO:1, SEQ ID NO:3, SEQ ID NO:12, or SEQ ID NO:13, in the sample indicates a diagnosis of glaucoma.

The present invention also provides a method for identifying agents potentially useful for treating glaucoma, by the steps:
a) obtaining cells expressing SAA (SEQ ID NO:1 or SEQ ID NO:2) or cells containing SAA promoter/reporter gene such that the reporter gene is expressed;
b) admixing a candidate substance with the cells; and
c) determining the level of SAA protein (SEQ ID NO:2 or SEQ ID NO:4) or the level of gene expression in the cells;
wherein an increase or decrease of the production of SAA protein or gene expression in the presence of said candidate substance indicates an agent potentially useful for the treatment of glaucoma.

In another aspect, the present invention provides a method for identifying an agent potentially useful for treating glaucoma, by the steps:
a) forming a reaction mixture comprising:
   (i) an SAA protein or a cell expressing SAA or a reporter gene driven by an SAA promoter;
   (ii) an SAA protein binding partner; and
   (iii) a test compound; and
b) detecting interaction of the SAA protein and binding partner or level of reporter gene products in the presence of the test compound and in the absence of the test compound;
wherein a decrease or increase in the interaction of the SAA protein with its binding partner in the presence of the test compound relative to the interaction in the absence of the test compound indicates a potentially useful agent for treating glaucoma.

In another aspect, the present invention provides a method for identifying an agent potentially useful for treating glaucoma, by the steps:
a) forming a reaction mixture comprising:
   (i) cells comprising SAA recombinant protein (SEQ ID NO:2 or SEQ ID NO:4) or cells comprising expression vectors comprising SEQ ID NO:1 or SEQ ID NO:3; and
   (ii) a test compound; and
b) detecting the effect on downstream signalling (IL-8) in the presence of the test compound and in the absence of the test compound;
   wherein a decrease or increase in the downstream signalling in the presence of the test compound relative to the interaction in the absense of the test compound indicates a potentially useful agent for treating glaucoma.

In preferred aspects, the cells containing the SAA protein or expression vectors will be HL-60 cells.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1. QPCR analysis of SAA expression in 12 glaucoma vs. 11 normal TM tissues. NTM and GTM represent average expression level of the gene in normal and glaucoma groups, respectively.

FIG. 2A. QPCR analysis of SAA expression in TM cell lines. NTM and GTM represent average expression level of the gene in normal and glaucoma groups, respectively.

FIG. 2B. QPCR analysis of SAA expression in optic nerve head tissues. NTM and GTM represent average expression level of the gene in normal and glaucoma groups, respectively.

FIG. 3. SAA protein in TM tissues from normal and glaucoma donors (n=6). A significant increase (3-fold) in SAA was observed in glaucoma TM tissues compared to normal tissue (p=0.05). The bars show mean+/−s.e.m.

FIG. 4. SAA protein determined by ELISA in human aqueous humor from normal and glaucomatous individuals. The values are expressed as the average SAA in ng/ml of aqueous humor, +/−s.e.m. (p=0.0001).

FIG. 5. IL-8 secretion by HL-60 cells in response to increasing concentrations of rhSAA.

FIG. 6. Effect of Adv.SAA2 on mouse IOP by intravitreal injection. IOP was measured with the rebound tonometers TonoLab®.

FIG. 7. SAA expression in mouse eyes from Balb/c mice 28 days after intravitreal injection. SAA was measured by ELISA. Control: Adv.null injected eyes and no injection eyes (contralateral eyes of Adv.SAA2 injected eyes; n=16); Adv.SAA: Adv.SAA2 injected eyes (n=17).

FIG. 8A and FIG. 8B. Effect of intravitreal injection of Ad.SAA2+ anti-CD40L antibody on Balb/c mouse IOP (FIG. 8A) and iris hyperemia (FIG. 8B). Data are presented as mean and SEM.

FIG. 9. Effect of recombinant human Serum Amyloid A (rhSAA, 1 μg/mL; treatment started at time 0) on IOP of perfused human anterior segments.

FIG. 10. Effect of recombinant human Serum Amyloid A (rhSAA, 1 μg/mL; treatment started at time 0) on interleukin-8 (IL-8) level in the perfusate of perfused human anterior segments.

FIG. 11. Effect of MAP p38 kinase inhibitors on induction of IL-8 by SAA treatment (1 μg/ml) in TM cells. A: Effect of SB203580 (4-(4-fluorophenyl)-2-(4-methylsulfinylphenyl)-5-(4-pyridyl)-1H-imidazole). B: Effect of 4-azaindole and BIRB-796 (1-(5-tert-butyl-2-p-tolyl-2H-pyrazol-3-yl)-3(4-(2-morpholin-4-yl-ethoxy)naph-thalen-1-yl)urea) at 50 μM. IL-8 was measured in the media by ELISA.

FIG. 12. Inhibition of SAA stimulated IL-8 secretion by SB203580 in TM cells and HL-60 cells. NTM650-03, p9 or HL-60 cells were treated in serum-free DMEM for 4 hours with 1 μg/ml SAA and the indicated concentrations of SB202580. IL-8 was measured in the media by ELISA. Calculated IC₅₀=15 μM in TM cells and 25 μM in HL-60 cells.

FIG. 13. Inhibition of SAA stimulated IL-8 secretion by the p38 MAP kinase inhibitors, 4-azaindole and BIRB-796 in HL60 cells treated in serum-free DMEM for 4 hours with 1 μg/ml SAA and the indicated concentrations of inhibitors. IL-8 was measured in the media by ELISA. Calculated IC₅₀: 4-azaindole=0.3 μM, BIRB-796=0.5 μM.

FIG. 14A and FIG. 14B. Effect of topical administration of 3-(4-fluorophenyl)-4-[2-(trans-4-hydroxy-4-methylcyclohexylamino)pyrimidin-4-yl]-1-(tetrahydropyran-4-yl)imidazolin-2-one at three concentrations on Ad.SAA induced OHT in mice. FIG. 14A: IOP data during the study. The viral vector was injected at Day 0. The mice were treated with 3-(4-fluorophenyl)-4-[2-(trans-4-hydroxy-4-methylcyclohexylamino)pyrimidin-4-yl]-1-(tetrahydropyran-4-yl)imidazolin-2-one or vehicle twice daily via topical ocular administration at the indicated days (horizontal bars). Conscious mouse IOP was monitored by the TonoLab rebound tonometer. Data are mean and SEM, n=7 for each of the Ad.SAA-injected groups; n=28 for the “No Injection” group. FIG. 14B presents the peak IOP-lowering effect of each concentration of 3-(4-fluorophenyl)-4-[2-(trans-4-hydroxy-4-methylcyclohexylamino)pyrimidin-4-yl]-1-(tetrahydropyran-4-yl)imidazolin-2-one in carbopol vehicle on Ad.SAA-induced TOP from the same study.

FIG. 15. Neuroprotective effect of 3-(4-fluorophenyl)-4-[2-(trans-4-hydroxy-4-methylcyclohexylamino)pyrimidin-4-yl]-1-(tetrahydropyran-4-yl)imidazolin-2-one in adult rat retinal ganglion cells (RGC)—glutamate-induced RGC cytotoxicity. Cells were cultured for 3 days in the absence (control) or presence of 100 μM L-glutamate. Cell survival was quantified by counting Thy-1-positive cells. *: p<0.01 vs. glutamate alone via one-way ANOVA, then Bonferroni's test.

FIG. 16. Effects of various test agents on the survival of cultured adult rat RGC. Cells were cultured for 3 days in the absence (control) or presence of TNFα (10 ng/mL). Cell survival was quantified by counting Thy-1-positive cells. *: p<0.01; **: p<0.05 vs. TNFα alone via one-way ANOVA, then Bonferroni's test.

FIG. 17. Neuroprotective effect of p38Ki in adult rat retinal ganglion cells—trophic factor withdrawal-induced RGC cytotoxicity. 3-(4-fluorophenyl)-4-[2-(trans-4-hydroxy-4-methylcyclohexylamino)pyrimidin-4-yl]-1-(tetrahydropyran-4-yl)imidazolin-2-one was protective against TFW at all tested concentrations. *: p<0.01; **: p<0.05 vs. TFW via One-way ANOVA, then Bonferroni's test.

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including proteinaceous plaque-like material. In addition, there are also changes that occur in the glaucomatous optic nerve head (ONH). In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

The present inventors have discovered that the expression of Serum Amyloid A (SAA) mRNA and protein are significantly upregulated in glaucomatous TM tissues and cells. The inventors have verified the differential mRNA expression seen using Affymetrix gene chips by real time quantitative polymerase chain reaction (QPCR) and increased SAA protein levels by SAA ELISA. This is the first time SAA has been shown to be expressed in the TM.

Human SAA comprises a number of small, differentially expressed apolipoproteins encoded by genes localized on the short arm of chromosome 11. There are four isoforms of SAAs. SAA1 (SEQ ID NO:2), encoded by SEQ ID NO:1, and SAA2 (SEQ ID NO:4), encoded by SEQ ID NO:3, are known as acute phase reactants, like C-reactive protein, that is, they are dramatically upregulated by proinflammatory cytokines. The 5′UTR promoter regions of SAA1 and SAA2 genes are also provided (SEQ ID NO:12 and SEQ ID NO:13, respectively). SAA3 (SEQ ID NO:5) is a pseudogene and SAA4 (SEQ ID NO:6) is a low level constitutively expressed gene encoding constitutive SAA4 (SEQ ID NO:7). SAA2 has two isoforms, SAA2α (SEQ ID NO:9), encoded by SEQ ID NO:8, and SAA2β (SEQ ID NO:11), encoded by SEQ ID NO:10, which differ by only one amino acid. SAA1 and SAA2 proteins are 93.5% identical at the amino acid level (SEQ ID NO:2 and SEQ ID NO:4, respectively) and these genes are 96.7% identical at the nucleotide level (SEQ ID NO:1 and SEQ ID NO:3, respectively).

SAA is an acute-phase reactant whose level in the blood is elevated approximately 1000-fold as part of the body's responses to various injuries, including trauma, infection, inflammation, and neoplasia. As an acute-phase reactant, the liver has been considered to be the primary site of expression. However, extrahepatic SAA expression was described initially in mouse tissues, and later in cells of human atherosclerotic lesions (O'Hara et al. 2000). Subsequently, SAA mRNA was found widely expressed in many histologically normal human tissues. Localized expression was noted in a variety of tissues, including breast, stomach, small and large intestine, prostate, lung, pancreas, kidney, tonsil, thyroid, pituitary, placenta, skin epidermis, and brain neurons. Expression was also observed in lymphocytes, plasma cells, and endothelial cells. SAA protein expression co-localized with SAA mRNA expression has also been reported in histologically normal human extrahepatic tissues. (Liang et al. 1997; Urieli-Shoval et al. 1998).

SAA isoforms are apolipoproteins that become a major component of high-density lipoprotein (HDL) in the blood plasma of mammals and displaces A-I (ApoA-I) and phospholipid from the HDL particles (Miida et al. 1999). SAA binds cholesterol and may serve as a transient cholesterol-binding protein. In addition, over-expression of SAA1 or SAA2 leads to the formation of linear fibrils in amyloid deposits, which can lead to pathogenesis (Uhlar and Whitehead 1999; Liang et al. 1997). SAA plays an important role in infections, inflammation, and in the stimulation of tissue repair. SAA concentration may increase up to 1000-fold following inflammation, infection, necrosis, and decline rapidly following recovery. Thus, serum SAA concentration is considered to be a useful marker with which to monitor inflammatory disease activity. Hepatic biosynthesis of SAA is up-regulated by pro-inflammatory cytokines, leading to an acute phase response. Chronically elevated SAA concentrations are a prerequisite for the pathogenesis of secondary amyloidosis, a progressive and sometimes fatal disease characterized by the deposition in major organs of insoluble plaques composed principally of proteolytically cleaved SAA. This same process also may lead to atherosclerosis. There is a requirement for both positive and negative SAA control mechanisms to maintain homeostasis. These mechanisms permit the rapid induction of SAA expression to fulfill host-protective functions, but they also must ensure that SAA expression is rapidly returned to baseline levels to prevent amyloidosis. These mechanisms include modulation of promoter activity involving, for example, the inducer nuclear factor kB (NF-kB) and its inhibitor IkB, up-regulation of transcription factors of the nuclear factor for interleukin-6 (NF-IL6) family, and transcriptional repressors such as yin and yang 1 (YY1). Post-transcriptional modulation involving changes in mRNA stability and translation efficiency permit further up- and down-regulatory control of SAA protein synthesis to be achieved. In the later stages of the AP response, SAA expression is effectively down-regulated via the increased production of cytokine antagonists such as the interleukin-1 receptor antagonist (IL-1Ra) and of soluble cytokine receptors, resulting in less signal transduction driven by pro-inflammatory cytokines (Jensen and Whitehead 1998).

There are several reports suggesting that primary amyloidosis may be associated with glaucoma. For example, it was found that amyloid was deposited in various ocular tissues including the vitreous, retina, choroid, iris, lens, and TM in primary systemic amyloidosis patients (Schwartz et al. 1982). Ermilov et al. (1993) reported that in 478 eyes of 313 patients, aged 25 years to 90 years, with cataracts, glaucoma, and/or diabetes mellitus, 66 (14%) of the eyes contained amyloid-pseudoexfoliative amyloid (PEA). Krasnov et al. (1996) reported that 44.4% of 115 patients with open-angle glaucoma revealed extracellular depositions of amyloid. Amyloidosis was revealed in the sclera in 82% of the cases and in the iris in 70% of the cases. A number of clinical conditions, including Alzheimer's disease, exhibit aberrant amyloid tissue deposits associated with disease. However, amyloids are molecularly heterogeneous and encoded by different amyloid genes. The previous reports are unclear regarding which amyloid(s) might be associated with glaucoma. The present inventors have shown, for the first time, that SAA gene expression is elevated significantly in glaucomatous TM tissues. Increased SAA may be involved in the generation of elevated IOP and damage to the optic nerve leading to vision loss in glaucoma patients. The present invention provides methods of using a finding of increased SAA expression to diagnose glaucoma. The present invention further provides methods for screening for agents that alter SAA expression or function in order to identify potentially anti-glaucomatous agents. In another aspect, the present invention provides methods and compositions of using agents that antagonize SAA actions and/or interactions with other proteins for the treatment of glaucoma.

### Diagnosing Glaucoma

Based on the inventors' finding that certain subjects with glaucoma have increased levels of SAA expression, the present invention provides a variety of methods for diagnosing glaucoma. Certain methods of the invention can detect mutations in nucleic acid sequences that result in inappropriately high levels of SAA protein. These diagnostics can be developed based on the known nucleic acid sequence of human SAA, or the encoded amino acid sequence (see Miller 2001). Other methods can be developed based on the genomic sequence of human SAA or of the sequence of genes that regulate expression of SAA. Still other methods can be developed based upon a change in the level of SAA gene expression at the mRNA level.

In alternative embodiments, the methods of the invention can detect the activity or level of SAA signaling proteins or genes encoding SAA signaling proteins. For example, methods can be developed that detect inappropriately low SAA signaling activity, including for example, mutations that result in inappropriate functioning of SAA signaling components, including SAA induction of IL-8. In addition, non-nucleic acid based techniques may be used to detect alteration in the amount or specific activity of any of these SAA signaling proteins.

A variety of means are currently available to the skilled artisan for detecting aberrant levels or activities of genes and gene products. These methods are well known by and have become routine for the skilled artisan. For example, many methods are available for detecting specific alleles at human polymorphic loci. The preferred method for detecting a specific polymorphic allele will depend, in part, upon the molecular nature of the polymorphism. The various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. A variety of methods are available for detecting the presence of a particular single nucleotide polymorphic allele in an individual. Advancements in the field have provided accurate, easy, and inexpensive large-scale SNP genotyping. For example, see U.S. Pat. No. 4,656,127; French Patent 2,650,840; PCT App. No. WO91/02087; PCT App. No. WO92/15712; Komher et al. 1989; Sokolov 1990; Syvanen et al. 1990; Kuppuswamy et al. 1991; Prezant et al. 1992; Ugozzoli et al. 1992; Nyren et al. 1993; Roest et al. 1993; and van der Luijt et al. 1994).

Any cell type or tissue may be utilized to obtain nucleic acid samples for use in the diagnostics described herein. In a preferred embodiment, the DNA sample is obtained from a bodily fluid, e.g., blood, obtained by known techniques (e.g. venipuncture), or buccal cells. Most preferably, the samples for use in the methods of the present invention will be obtained from blood or buccal cells. Alternately, nucleic acid tests can be performed on dry samples (e.g. hair or skin).

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo 1992).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an SAA signaling component that is indicative of glaucoma and having about 5, 10, 20, 25 or 30 contiguous nucleotides around the mutation or polymorphic region. In a preferred embodiment of the invention, several probes capable of hybridizing specifically to other allelic variants involved in glaucoma are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996). In one embodiment, a chip comprises all the allelic variants of at least one polymorphic region of a gene. The solid phase support is then contacted with a test nucleic acid and hybridication to the specific probes is detected. Accordingly, the identity of numerous allelic variants of one or more genes can be identified in a simple hybridization experiment.

These techniques may further include the step of amplifying the nucleic acid before analysis. Amplification techniques are known to those of skill in the art and include, but are not limited to, cloning, polymerase chain reaction (PCR), polymerase chain reaction of specific alleles (ASA), ligase chain reaction (LCR), nested polymerase chain reaction, self sustained sequence replication (Guatelli et al. 1990), transcriptional amplification system (Kwoh et al. 1989), and Q-Beta Replicase (Lizardi, et al. 1988).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, SSCP, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of SAA that is indicative of glaucoma under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, aberrant levels or activities of SAA that are indicative of glaucoma are identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed my Maxim and Gilbert (1977) or Sanger (1977). It is also contemplated that any of a variety of automated sequencing procedures may be utilized when performing the subject assays, including sequencing by mass spectrometry (see, for example WO94/16101; Cohen et al. 1996; Griffin et al. 1993). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamin or osmium tetraoxide and with piperidine) can be used to detect mismatched bases in RNA/RNA or RNA/DNA or DNA/DNA heteroduplexes (Myers et al. 1985b; Cotton et al. 1988; Saleeba et al. 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T and G/T mismatches (Hsu et al. 1994; U.S. Pat. No. 5,459,039).

In other embodiments, alterations in electrophoretic mobility will be used to identify aberrant levels or activities of SAA that are indicative of glaucoma. For example, single strand conformation polymorphism (SSCP) may be used to detect differences in electrophoretic mobility between mutant and wild type nucleic acids (Orita et al. 1989; Cotton 1993; Hayashi 1992; Keen et al. 1991).

In yet another embodiment, the movement of alleles in polyacrylamide gels containing a gradient of denaturant is assayed using denaturing gradient gel electrophoresis (DGGE) (Myers et al. 1985a). In a further embodiment, a temperature gradient is used in place of a denaturing agent gradient to identify differences in the mobility of control and sample DNA (Rosenbaum and Reissner 1987).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. 1986; Saiki et al. 1989). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al. 1989) or at the extreme 3′ end of one primer where, under appropriate conditions, mismatch can prevent, or reduce polymerase extension (Prossner 1993). In addition it may be desirable to introduce a novel restriction site in the region of the mutation to create cleavage-based detection (Gasparini et al. 1992). It is anticipated that in certain embodiments amplification may also be performed using Taq ligase for amplification (Barany 1991). In such cases, ligation will occur only if there is a perfect match at the 3′ end of the 5′ sequence making it possible to detect the presence of a known mutation at a specific site by looking for the presence or absence of amplification.

In another embodiment, identification of an allelic variant is carried out using an oligonucleotide ligation assay (OLA), as described, E.g., in U.S. Pat. No. 4,998,617 and in Landegren et al. 1988). Nickerson et al. have described a nucleic acid detection assay that combines attributes of PCR and OLA (Nickerson et al. 1990). In this method, PCR is used to achieve the exponential amplification of target DNA, which is then detected using OLA.

Several techniques based on this OLA method have been developed and can be used to detect aberrant levels or activities of SAA that are indicative of glaucoma. For example, U.S. Pat. No. 5,593,826 and To be et al. (1996), describe such techniques that are frequently used.

In one embodiment, fenofibrate, a peroxisome proliferator-activated receptor α (PPARα) agonist, may be formulated in a pharmaceutically acceptable composition and used to treat glaucoma by modulating SAA expression. Studies have shown that fenofibrate and WY 14643 treatment reduces plasma SAA concentration (Yamazaki et al. 2002). It is believed that other PPARα agonists, such as ciprofibrate, 2-bromohexadecanoic acid, bezafibrate, ciprofibrate and ciglitizone may also be useful for the treatment of glaucoma.

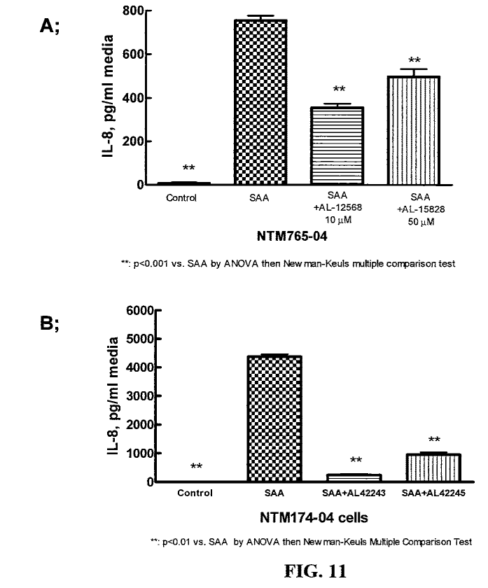

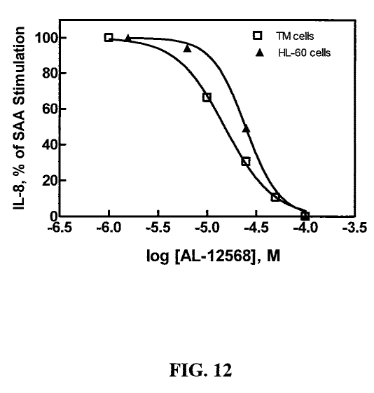

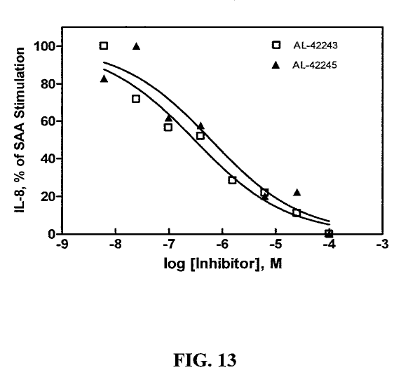

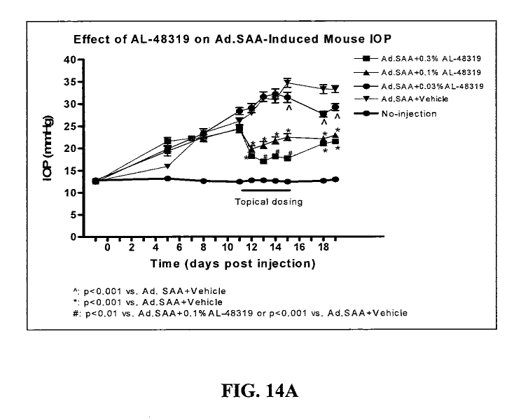

In another embodiment, p38 MAP kinase inhibitors may be used to treat glaucoma by modulating SAA induced expression of IL-8 and downstream signaling events. The inventors showed that SAA stimulates secretion of IL-8 in trabecular meshwork cells and tissue. One pathway for upregulation of IL-8 is through activation of MAP kinases. The inventors further showed that inhibitors of p38 MAP kinase block SAA induction of IL-8 in TM cells, in perfusion cultured human eyes, and in vivo in rodent eyes. Compounds representing different classes of MAPK inhibitors in TM cells were found to be effective inhibitors of SAA induced IL-8 expression (Table 3). The most potent of these were the p38 MAPK inhibitors, SB203580, 4-azaindole, and BIRB-796 (FIG. 11). Dose response curves for SB203580 inhibition of SAA induced IL-8 generated in both TM cells and HL60 cells gave similar results (IC₅₀=15 μM in TM cells and 25 μM in HL60 cells, FIG. 12). Inhibition curves for 3-(4-fluorophenyl)-2-(pyridin-4-yl)-1H-pyrrolo[3,2-b]pyridine (also referred to herein as a 4-azaindole) and BIRB0796 conducted in HL60 cells showed both of these compounds to be approximately 10 times more effective than SB203580 (IC₅₀=0.3 for 4-azaindole and 0.5 μM for BIRB-796 (FIG. 13).

TABLE 3
Compound Classes Screened for Inhibition of SAA Induced IL-8
Secretion in TM Cells

| | | | % Inhibition of SAA induced IL-8 | | | | |
|---|---|---|---|---|---|---|---|
| | | | secretion | | | | |
| Compound | Target | Selectivity | 100 μM | 50 μM | 25 μM | 10 μM | 1 μM |
| SB203580 | MAPK | p38 MAP kinase | 98.7 | 73.8 | 61.0 | 38.1 | 16.3 |
| SB202190 | MAPK | p38 MAP kinase | | 38.2 | | 43.3 | 17.2 |
| BIRB-796 | MAPK | p38 MAP kinase | | 78.1 | | 28.9 | |
| 4-azaindole | MAPK | p38 MAP kinase | | 94.3 | | 22.6 | |
| PD98059 | MAPK | MEK | | 34.0 | | | |
| U0126 | MAPK | MEK | | 53.0 | | 50.6 | 30.9 |
| Fenofibrate | SAA | PPARα agonist | | 57.2 | | 52.7 | 59.9 |

Chemical names for the compounds identified in Table 3 are as follows:
SB203580: 4-(4-fluorophenyl)-2-(40-methylsulfinylphenyl)-5-(4-pyridyl)-1H-imidazole;
SB202190: 4-[4-(4-fluorophenyl)-5-(4-pyridinyl)-1H-imidazol-2-yl]phenol;
BIRB-796: 1-(5-tert-butyl-2-p-tolyl-2H-pyrazol-3-yl)-3(4-(2-morpholin-4-yl-ethoxy)naph-thalen-1-yl)urea;
4-azaindole: 3-(4-fluorophenyl)-2-(pyridin-4-yl)-1H-pyrrolo[3,2-b]pyridine;
PD98059: 2′-amino-3′-methoxyflavone;
U0126: 1,4-diamino-2,3-dicyano-1,4-bis(2-aminophynyltio)butadiene;
CalBio506126: 2-(4-chlorophenyl)-4-(4-fluorophenyl)-5-pyridin-4-yl-1,2-dihydropyrazol-3-one

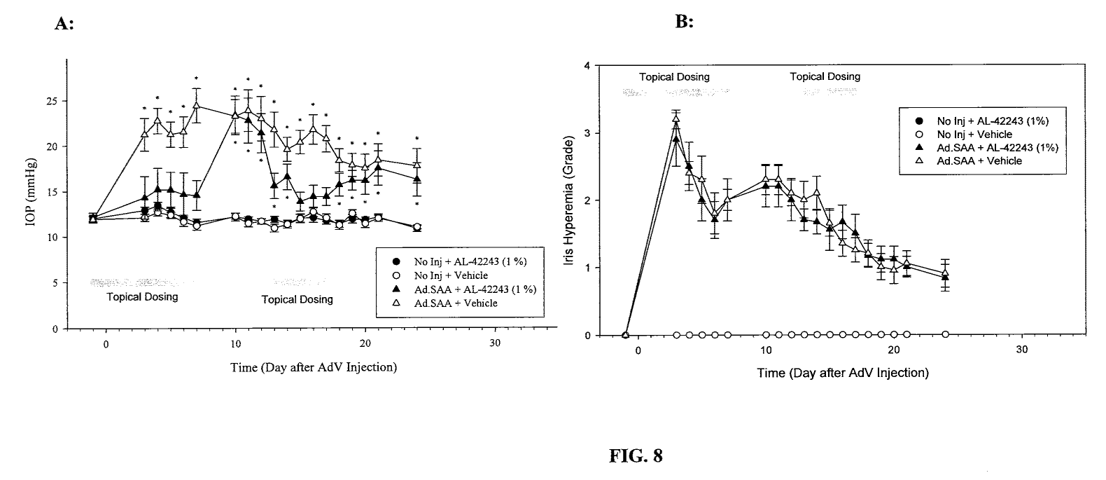

In vitro assays showed that several p38MAPK inhibitors significantly blocked IL-8 induction by SAA. 3-(4-fluorophenyl)-2-(pyridine-4-yl)-1H-pyrrolo-[3,2-b]pyridine, one of most potent p38 MAPK inhibitors was evaluated for lowering IOP in the mouse by topical application of a 1% suspension after intravitreal injection of Ad.SAA2 (2×10⁷ pfu/eye)+antiCD40L. Adv.SAA2, caused a significant IOP elevation, which peaked at days 10-12 with 10-12 mmHg increase from baseline, followed by a slow decline. At day 24, the IOP was 6-7 mmHg above baseline. The ocular hypertension was blocked by topical administration of 4-azaindole (1%; b.i.d.). After 4-azaindole administration was stopped at Day 7, IOP returned to the same level as the vehicle-treated Ad.SAA2-injected group. When drug administration was resumed at day 13, IOP was again lowered to baseline in 3 days (FIG. 8). This experiment was repeated and similar results were obtained. The in vivo data showed that the p38MAPK inhibitor, 4-azaindole, can counteract ocular hypertension induced by Ad.SAA2.

Preferred compounds for this embodiment of the invention are those classes of compounds listed in Table 3, and extends to additional classes of compounds that exibit inhibitory properties for p38MAP kinases as described in these examples and include SB202190, SB203580, SB220025, PD 169316, SB 239063, 4-azaindole, BIRB-796, CalBio506126; and compounds disclosed in WO2005/105790, such as compounds described in Examples 1-136 and compounds I-1 through I-69. For example, the compound 3-(4-fluorophenyl)-4-[2-(trans-4-hydroxy-4-methylcyclohexylamino)pyrimidin-4-yl]-1-(tetrahydropyran-4-yl)imidazolin-2-one would be useful in the methods of the present invention.

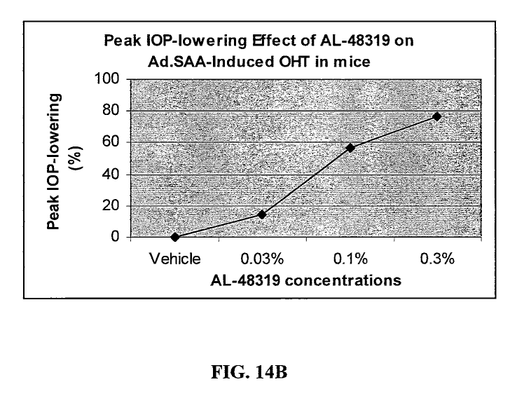

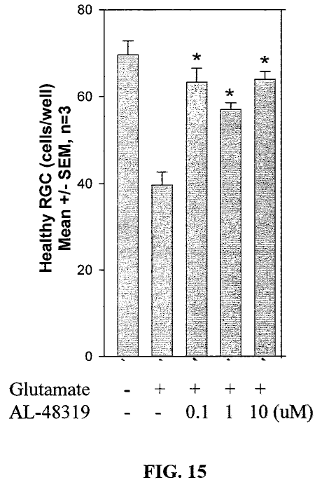

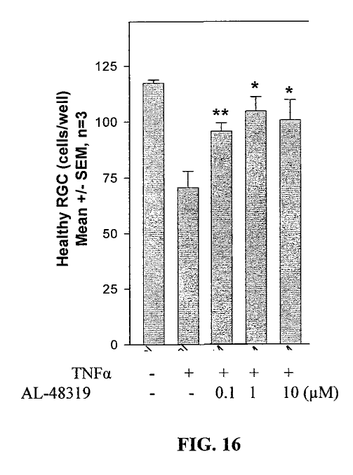

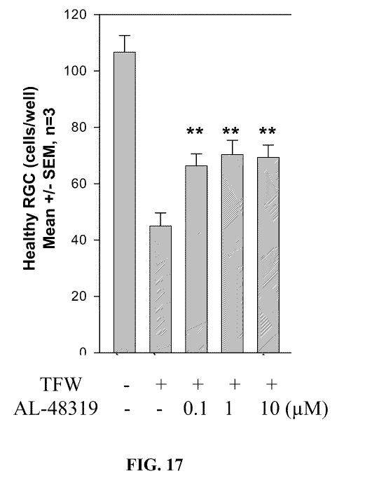

The present inventors have shown that compound 3-(4-fluorophenyl)-4-[2-(trans-4-hydroxy-4-methylcyclohexylamino)pyrimidin-4-yl]-1-(tetrahydropyran-4-yl)imidazolin-2-one (AL-48319), delivered topically to the eye, effectively lowers IOP in the Ad.SAA2-induced ocular hypertension mouse model (FIG. 14). The compound 3-(4-fluorophenyl)-4-[2-(trans-4-hydroxy-4-methylcyclohexylamino)pyrimidin-4-yl]-1-(tetrahydropyran-4-yl)imidazolin-2-one is among the preferred compounds disclosed in WO2005/105790. The present inventors also demonstrated that 3-(4-fluorophenyl)-4-[2-(trans-4-hydroxy-4-methylcyclohexylamino)pyrimidin-4-yl]-1-(tetrahydropyran-4-yl)imidazolin-2-one is an effective neuroprotectant in rat RGC culture models (FIG. 15; FIG. 16; FIG. 17).

The present inventors further postulate that agents that prevent amyloid-induced cell death may be useful for protecting TM and other ocular cells in the anterior uvea and at the back of the eye, especially the retina and optic nerve head.

The Compounds of this invention, can be incorporated into various types of ophthalmic formulations for delivery to the eye (e.g., topically, intracamerally, or via an implant). The Compounds are preferably incorporated into topical ophthalmic formulations for delivery to the eye. The Compounds may be combined with opthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a Compound in a physiologically acceptable isotonic aqueous buffer. Further, the ophthalmic solution may include an opthalmologically acceptable surfactant to assist in dissolving the Compound. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the Compound in a hydrophilic base prepared from the combination of, for example, carbopol-974, or the like, according to the published formulations for analogous ophthalmic preparations; preservatives and tonicity agents can be incorporated.

The Compounds are preferably formulated as topical ophthalmic suspensions or solutions, with a pH of about 4 to 8. The establishment of a specific dosage regimen for each individual is left to the discretion of the clinicians. The Compounds will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The Compounds can also be used in combination with other agents for treating glaucoma, such as, but not limited to, β-blockers, prostaglandins, carbonic anhydrase inhibitors, α₂ agonists, miotics, and neuroprotectants.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Increased Expression of SAA1 and SAA2 in Glaucomatous TM Cells and Tissues

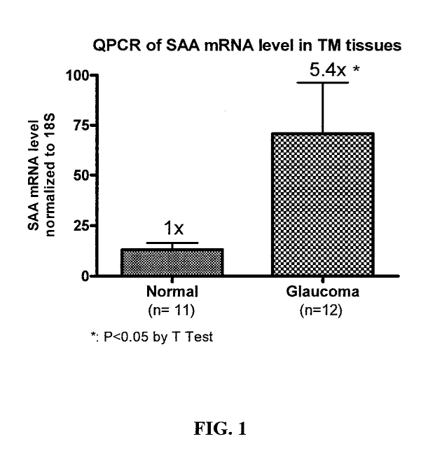

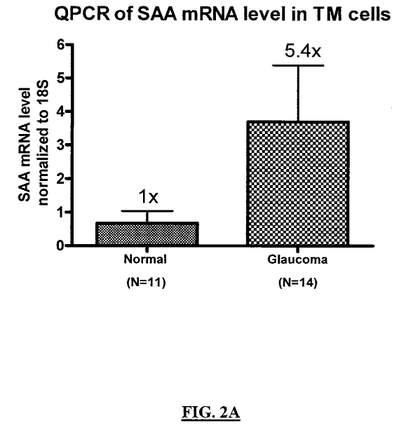

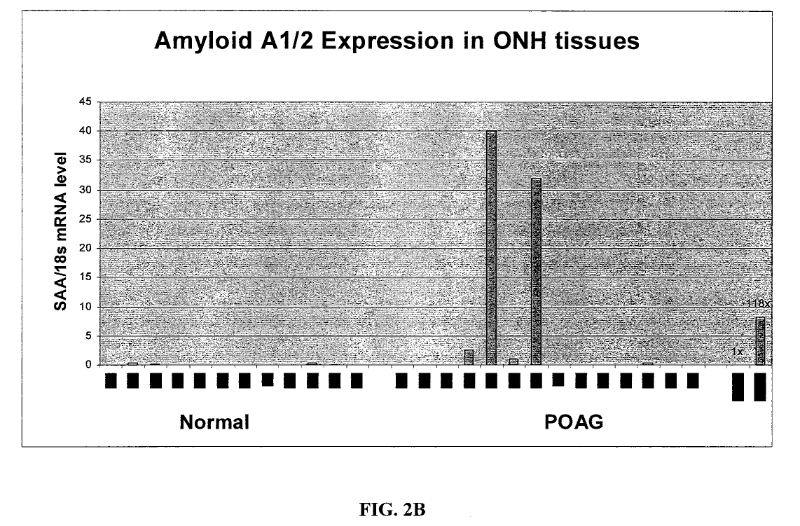

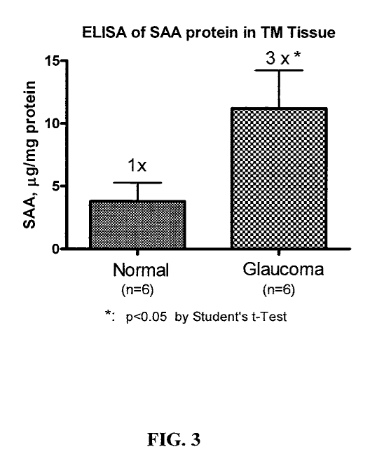

RNA pools of TM tissues from 13 normal donors vs. 9 glaucoma donors was used to determine gene expression using the Affymetric GeneChips set (HG-U133). Amyloid A2 expression was identified to increase 4 fold in glaucoma comparing to that in normal TM tissues. To confirm this result, QPCR was conducted using individual RNA from 12 glaucoma and 11 normal TM tissues. Five from 12 glaucoma TM tissues (42%) showed significant increase in SAA1/2 expression. Average of SAA expression in the 12 glaucoma TM was 5.4 fold to that in the 11 normal TM (FIG. 1). In addition, a similar trend of SAA differential expression was observed in glaucoma TM cells or glaucoma optic nerve head tissues. There was an average increase of 5.4-fold in glaucoma TM cells (14 glaucoma vs. 11 normal TM cell lines, FIG. 2A) and 118-fold in glaucoma optic nerve head tissues (14 glaucoma vs. 12 normal, FIG. 2B) compared to normals, respectively. ELISA of SAA in TM tissues from 6 normal and 6 glaucoma donors showed that SAA protein was also significantly increased in glaucoma TM tissues compared to normals. There was a 3-fold difference in SAA concentration in glaucomatous tissue compared to normal tissue (11.3 and 3.8 μg/mg protein respectively). These data are shown in FIG. 3.

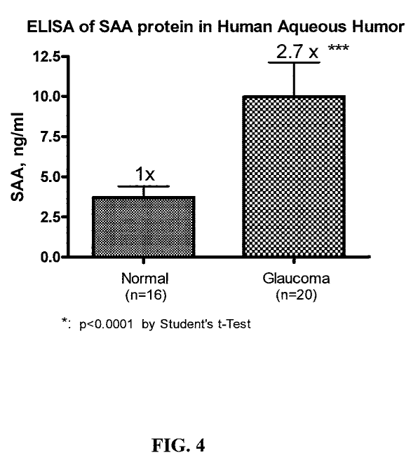

An association of increased expression of SAA with glaucoma was further demonstrated in human aqueous humor. SAA protein was measured by ELISA in aqueous humor from 16 normal and 20 glaucomatous individuals. SAA was found to be almost 3 times higher in glaucomatous aqueous humor than in normal samples (10.0 ng/ml vs. 3.7 ng/ml respectively). The results are shown in FIG. 4.

### Example 2

### Formulation of Fenofibrate for Topical Application

1% Fenofibrate suspension for topical application to decrease SAA and lower IOP in the eye.

| Description | Conc. | Units | Purpose |
|---|---|---|---|
| Fenofibrate, NOC | 1% | W/V % | active ingredient |
| hydroxypropyl | 0.5% | W/V % | viscosity modifier |
| methylcellulose | | | (2910) (E4M), USP |
| dibasic sodium phosphate | 0.2% | W/V % | buffering agent |
| | | | (anhydrous), usp |
| sodium chloride, usp | 0.75% | W/V % | tonicity agent |
| disodium edta | 0.01% | W/V % | chelating agent |
| | | | (edetate |
| | | | disodium), usp |
| polysorbate 80, nf | 0.05% | W/V % | wetting agent |
| benzalkonium chloride, nf | 0.01% | W/V % | preservative |
| sodium hydroxide, nf | q.s. pH | W/V % | pH adjust |
| hydrochloric acid, nf | q.s. pH | W/V % | pH adjust |
| purified water, usp | q.s. 100% | W/V % | vehicle |

### Example 3

### Procedure for Screening and Identifying Compounds that Alter the Expression of SAA mRNA or SAA Proteins

One method that can be used for screening for agents that alter SAA expression and function is to determine changes in SAA protein levels. Kits for in vitro assay for quantitative determination of Serum Amyloid A (SAA) in animal or human sera, plasma, buffered solutions, cell culture media, and tissue or cell extracts are commercially available. The assay is a solid phase sandwich Enzyme Linked-Immuno-Sorbent Assay (ELISA). A monoclonal antibody specific for SAA has been coated onto the wells of a microtiter plate. Samples, including standards of known SAA content, or unknowns, are added to these wells along with a secondary antibody conjugated to alkaline phosphatase or peroxidase. The antibodies are constructed such that neither one interferes with the binding epitope of the other. The SAA is both captured on the plate by the immobilized antibody and labeled with the conjugated second antibody in a one step procedure. After an incubation period, the plate is washed to remove all unbound material and a substrate (PNPP or peroxide) is added. The intensity of the colored product is proportional to the concentration of SAA present in the unknown sample.

### Example 4

### Induction of SAA in Cultured Cell Lines for Screening Compounds that Alter the Expression of SAA mRNA or Protein

The human hepatoma cell line, HepG2, is widely used for studies on SAA induction by cytokines, for transfection with plasmids, and reporter assays. SAA mRNA and protein synthesis can be induced by various cytokines in several human hepatoma cell lines including PCL/PRF/5, HepB and HepG2 (Uhlar and Whitehead 1999). SAA synthesis by human aortic smooth muscle cells (HASMC) is induced by glucocorticoid hormones and not by the proinflammatory cytokines, IL-1, IL-6, and TNF-α, which stimulate the production of SAA by hepatocytes (Kumon et al. 2002b; Kumon et al. 2001; Thorn and Whitehead 2002). SAA stimulated the chemotactic migration of HASMC in a dose dependent manner when assayed using a Chemotaxicell culture chamber (Kumon et al. 2002a). SAA mRNA expression and protein production was demonstrated in primary cultures of rheumatoid arthritis synoviocytes (O'Hara et al. 2000).

### Example 5

### Functional Analysis of SAA in Cultured Cells

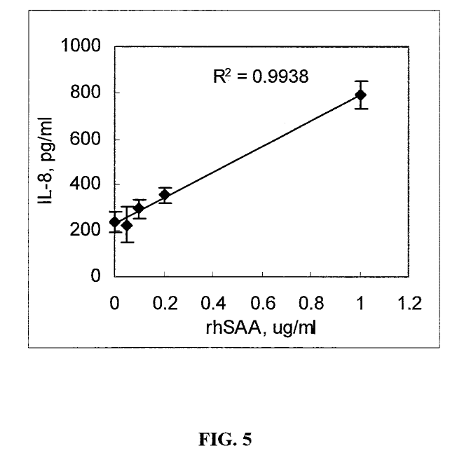

Cytokine-like properties of SAA include induction of IL-8 secretion by neutrophils. (Furlaneto and Campa, 2002; He et al. 2003). HL-60 cells, a promyelocytic cell line, was identified that responds to SAA with increased IL-8 secretion, and can be used for in vitro assays of SAA function. HL-60 cells were treated for four hours with increasing concentrations of recombinant human SAA, and IL-8 was measured in the media by ELISA. IL-8 secretion increased in a dose dependent manner (FIG. 5). HL-60 cells can be used as a surrogate cell line for functional assays to identify agents that alter SAA function and expression levels.

### Example 6

### Adenovirus Mediated SAA Expression Increases IOP and p38 MAPK Inhibitor Decreases the Induced IOP in the Mouse

The ability of upregulated SAA expression through adenovirus to elevate IOP and efficacy of p38 MAPK inhibitors in blockage of the induced IOP in mouse was studied.

### 1. Upregulated SAA Expression Elevates IOP in Mouse

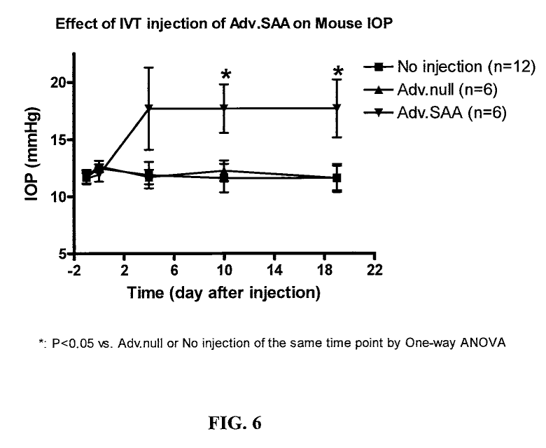

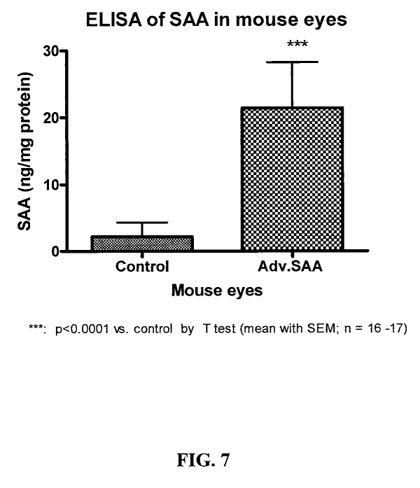

One eye of each Balb/c mice was intravitreally injected with Adv.SAA2 (treatment) or Adv.null (vehicle) at dosage of 7×10⁷ pfu/eye/2 ul. Contralateral eye of each animal was not injected. In addition to Adv, each animal received IP injection of anti-CD40L (0.5 mg/injection) on days −1,0,1, 2, 5, 9, & 14 to prolong the expression period of the Adv.SAA2. Mouse IOP was measured by Tonolab in a mask way. Mean of IOP for each eye was obtained from 18 to 30 measurements. Intravitreal injection of Adv.SAA2 in mice significantly increased IOP (49% or 5.8 mm Hg, n=6-8, p<0.05) (FIG. 6). SAA expression was significantly higher in all Adv.SAA2 treated eyes than that in control eyes including vehicle treated eyes and contralateral eyes without injection (p<0.0001; n=16) (FIG. 7). These results demonstrated that upregulating SAA expression can increase mouse IOP, providing evidence of SAA linkage to glaucoma pathogenesis.

### 2. p38 MAPK Inhibitor Lowers Adv.SAA2 Induced IOP in Mouse

After establishing that 4-azaindole, a P38 MAPK inhibitor inhibits the SAA-induced IL-8 expression in vitro, the inventors tested the effect of the compound on mouse IOP after intravitreal injection of Ad.SAA2 (2×10⁷ pfu)+antiCD40L by topical administration of 5 μL of 1% 4-azaindole or vehicle on both eyes, b.i.d., from day −1 to day 7 and day 13 to day 17. Again, intravitreal injection of Adv.SAA2 significantly increased mouse IOP from days 4 to 24 (vehicle group). Topical dosing of 4-azaindole significantly inhibited the Adv.SAA2-induced IOP during the period of treatment time (days −1 to 7 and days 13 to 17. 4-azaindole did not affect IOP in the non-injected eyes (FIG. 8A). Iris hyperemia was observed in all Ad.SAA2-injected eyes after day 4 and slowly decreased during the second week after injection (FIG. 8B). 4-azaindole did not affect hyperemia in the injected eyes, indicating the IOP-lowing effect of 4-azaindole was not through dispelling of iris hyperemia. These results demonstrate the potential of p38 MAPK inhibitors for treatment of ocular hypertension.

In another experiment, mice were treated with 3-(4-fluorophenyl)-4-[2-(trans-4-hydroxy-4-methylcyclohexylamino)pyrimidin-4-yl]-1-(tetrahydropyran-4-yl)imidazolin-2-one or vehicle by twice daily topical ocular administration at days 11, 12, 13, 14, and 15 (FIG. 14A). Conscious mouse IOP was monitored by TonoLab rebound tonometer. Data presented in FIG. 14A are mean and SEM, n=7 for each of the Ad.SAA-injected groups; n=28 for the “No injection” group. The dose-response study was completed. Three concentrations of 3-(4-fluorophenyl)-4-[2-(trans-4-hydroxy-4-methylcyclohexylamino)pyrimidin-4-yl]-1-(tetrahydropyran-4-yl)imidazolin-2-one (0.01%, 0.1% and 0.3%) in carbopol vehicle were evaluated in the Ad.SAA-induced OHT mouse model. This study showed that topical application (BID) of 3-(4-fluorophenyl)-4-[2-(trans-4-hydroxy-4-methylcyclohexylamino)pyrimidin-4-yl]-1-(tetrahydropyran-4-yl)imidazolin-2-one for 5 days lowered Ad.SAA-induced OHT in a dose dependent way (FIG. 14B). The maximum IOP decrease is 77% by 0.3% 3-(4-fluorophenyl)-4-[2-(trans-4-hydroxy-4-methylcyclohexylamino)pyrimidin-4-yl]-1-(tetrahydropyran-4-yl)imidazolin-2-one.

### Example 7

### Recombinant SAA Decreased Outflow Facility in Perfusion Cultured Human Eyes

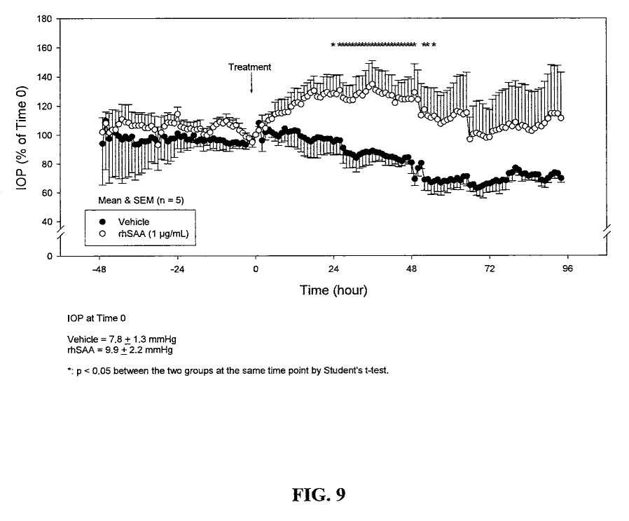

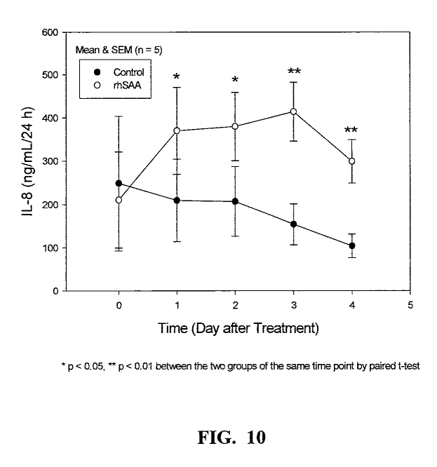

Five pairs of human eyes were perfused with media containing either recombinant SAA (1 μg/mL) (experimental eye) or an equivalent volume of vehicle (control eye). At the end of the culture period, 4 quadrants of each eye were examined by transmission electron microscopy to determine TM tissue viability. Perfusate of each eye was collected and used for ELISA measurement of IL-8 level. All five had an elevated IOP within 24 h of treatment (FIG. 9). All five had an elevated IL-8 level (FIG. 10). The change in IOP correlated with SAA-induced increase in interleukin-8 in the perfusates. All five pairs of eyes had acceptable post-perfusion TM viability scores. These results demonstrated that increased SAA level can elevate IOP in perfusion cultured human eyes.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### OTHER PUBLICATIONS

Furlenato, C J, and Campa A, A novel function of serum amyloid A: a potent stimulus for the release of tumor necrosis factor-alpha, interleukin-1 beta, and interleukin-8 by human blood neutrophil, BIOCHEM. BIOPHYS. RES. COMMUN 268:405-408 (2002).
He, R, Sang H, Ye, R D, Serum amyloid A induces IL-8 secretion through a G protein-coupled receptor, FPRL1/LXA4R, BLOOD 101:1572-1581 (2003).
Jensen L E and Whitehead A S, BIOCHEM. J. 334:489-503 (1998).
Jordat M S, et al., PLANTA MED. 68:667-71 (2002).
Kane et al., J. NEUROCHEM., 72: 1939-1947 (1999).
Kumon, Y., Hosokawa, T., Suehiro, T., Ideda, Y., Sipe, J. D., and Hashimoto, K., Acute-phase, but not constitutive serum amyloid A (SAA) is chemotactic for cultured human aortic smooth muscle cells, AMYLOID 9:237-241 (2002a).
Kumon, Y., Suehiro, T., Faulkes, D. J., Hosakawa, T., Ideda, Y., Woo, P., Sipe, J. D., and Hashimoto, K., Transcriptional regulation of Serum Amyloid A1 gene expression in human aortic smooth muscle cells involves CCAAT/enhancer binding proteins (C/EBP) and is distinct from HepG2 cells, SCAND. J. IMMUNOL. 56:504-511 (2002b).
Kumon, Y., Suehiro, T., Hashimoto, K., and Sipe, J. D., Dexamethasone, but not IL-1 alone, upregulates acute-phase serum amyloid A gene expression and production by cultured human aortic smooth muscle cells, SCAND J. IMMUNOL, 53:7-12 (2001).
Lambert et al., PROC. NAT. ACAD. SCI. USA 95: 6448-6453 (1998).
Liang, J. S., Sloane, J. A., Wells, J. M., Abraham, C. R., Fine, R. E., and Sipe, J. D., Evidence for local production of acute phase response apolipoprotein serum amyloid A in Alzheimer's disease brain, NEUROSCI. LETT. 225:73-76 (1997).
Liu et al., J. NEUROCHEM. 69: 2285-2293 (1997).
Miida T., Yamada, T., Yamadera, T., Ozaki, K., Inano, K., Okada, M., Serum amyloid A protein generates pre-beta 1 high-density lipoprotein from alpha-migrating high-density lipoprotein, BIOCHEM. 38(51):16958-16962 (1999).
Miller, Genome Biology 3(1):reviews 3001.1-3001.15 (2001) (also at http://genomebiology.com/2001/3/1/reviews/3001.1)
Nakagami et al., EUR. J. PHARMACOL. 457: 11-17 (2002a).
Nakagami et al., BR. J. PHARMACOL., 137: 676-682 (2002b).
O'Hara, R., Murphy, E. P., Whitehead, A. S., FitzGerald, O., and Bresnihan, B., Acute-phase serum amyloid A production by rheumatoid arthritis synovial tissue, ARTHRITIS RES. 2:142-144 (2000).
Pike et al., J. NEUROSCI. 13: 1676-1687 (1993).
Thorn, C. F. and Whitehead, A. S., Differential glucocorticoid enhancement of the cytokine-driven transcriptional activation of the human actue phase serum amyloid A genes, SAA1 and SAA, J. IMMUNOL. 169:399-406 (2002).
Uhlar, C. M., and Whitehead, A.S., Serum amyloid A, the major vertebrate acute-phase reactant, EUR. J. BIOCHEM. 265:501-523 (1999).
Urieli-Shoval, S., Cohen, P., Eisenberg, S., and Matzner, Y., Widespread expression of serum amyloid A in histologically normal human tissue. Predominant localization to the epithelium, J. HISTOCHEM. CYTOCHEM. 46:1377-1384 (1998).
Yamazaki et al., BIOCHEMICAL AND BIOPHYSICAL RES. COMM., 290:1114-1122 (2002).
Yankner et al., SCIENCE 250: 279-282 (1990)
Zhang et al., NEUROSCI. LETT. 312: 125-128 (2001)

We claim:

1. A method for treating glaucoma, said method comprising administering to a patient in need thereof a therapeutically effective amount of a composition comprising an inhibitor of p38 MAPkinase, wherein said p38 MAPkinase inhibitor is 3-(4-fluorophenyl)-4-[2-(trans-4-hydroxy-4-methylcyclohexylamino)pyrimidin-4-yl]-1-(tetrahydropyran-4-yl)imidazolin-2-one or a pharmaceutically acceptable salt thereof.

2. A method for lowering intraocular pressure in a patient suffering from elevated intraocular pressure, said method comprising administering to a patient in need thereof a therapeutically effective amount of a composition comprising an inhibitor of p38 MAPkinase, wherein said p38 MAPkinase inhibitor is 3-(4-fluorophenyl)-4-[2-(trans-4-hydroxy-4-methylcyclohexylamino)pyrimidin-4-yl]-1-(tetrahydropyran-4-yl)imidazolin-2-one or a pharmaceutically acceptable salt thereof.


SEQUENCE LISTING (13 sequences)

SEQ ID NO: 1 - DNA, 369 bp, homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 2 - protein, 122 aa, homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 3 - DNA, 570 bp, homo sapiens
agggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactgagcttcctcttcactctgctctcaggagacctggctatgaggccctcggggcagggatacaaagttagtgaggtctatgtccagagaagctgagatatggcatataataggcatctaataaatgcttaagaggtcaaaaaaaaaaaaaaaaaaaaaaaaaaaaaa

SEQ ID NO: 4 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGHGAEDSLADQAANKWGRSGRDPNHFRPAGLPEKY

SEQ ID NO: 5 - DNA, 4286 bp, homo sapiens
gatggttgacaactcccctcctcttccccctcttctactgtctactcctgggaccaagtgagccacgccagctcagatactacactgaccacagggaatcccaccttttccaaggaatggaagttgtgtagggaatattcaaatgttgcttagcattgccttagataagaaccaaagggacagggaaatcctctgacagctatctgccttataactttcattttactgtgcctaaaatatgctcagaacccagaaagaggcataattcctaattttggcaggctctaatctaaaataatgattctcaaacatggtgtgacttttgtctatttgctttatcctgggtcactgctcctcttctgtcagatactgggattccaatgagacaaatggaaatggagacgtagaccctctgaccttctatcttttatctatacacatacacctgtgtgtgtgtgtgtgtgtgtgtgtgtgcgtgtgtaaaaccgagtgggtttttttcttggaatgaaagaatggactaacattacaaaaaataaaaacttgaaacagaatgtgtattatccttggttgtgtttccttggccctgcagcaggatgaagctctccactggcatcattttctgctccctggtcctgggtgtcagcagccaaggatggttaacattcctcaaggcagctggccaaggtgaggtccacaggatagggggcaggaggctgcttctggctgcccccaggatgcagctgagcagaggccacatccccactgggcaaaggtgctagtgatgccacagatggatagagaaggggcatggtttttcataagcgtggttcctcatgcttttctggacagctttgacactcttctatgaggatcctccagccgaggtcgcataaggtgtgagctgcctcttttcagcaggaccatgagagagatgtggagttgaggggtgcatgttcccataataccggtggggctctactgccccctagtgggaaatctgggacagttcatgtctatgtctcctgggaagccaggaagcaggtggatcaaaagtgtgaggcgagtccatggggaagctgaacggagccaaccgtccccataaaaacaaccaagcttagctgagattttaatacgtactaggcactgtttaaatgtactaatgaattggtttccatcatttagtcctatgatgcaagcagcattatcccttaacagagaagctaacacacacacacacacacacacactaacacacacacacacacacacacacacacaaaccccaagatacgtaaagaagttccaaagcagagcaggattaacccaggcagtcttgctctgcagaacttgctcttaatcaaggtactctgctgctttcaaaacaagagtttcggatttgtgaacacatagctcatcctttatctaagaaatggcaaataggatgtggtgcctttggaaggtaagtctagctccacttatcccagtaaaacctacagtgaattaccttgatggtggttctactggggcttatatatggccaggaaactgctagcaagagaaatataccccgagggctgggcacagtggctcacacctgtaatcccagcactttgggaggctgaggtgggcagatcacctgaggtcaagagttcgagaccagcctggccaacatggcgaaatcctgtctctactaaaaatacagaaattagccgggtgtggtggcatgcgcctataatcccagcctctcgggaggctgagggagaagaattgcttgaactcaggaggcagaggttgcagtgagctgtgatcacaccactgcactccagcctaggagacagagcaagactccatctagagagacagagagagagagagagggagaaatataccccactagccataataaagtggcaaaattttgttttcagaatgcagtattttaaatttcaggtattattatttttctgagtctctgaaaaatggttttaaggatttgcttttaatcctatttacatgttcacacactcaactacaaatatctttcattccttaggttaatatttttcaaagggttgttctgggaccacttgcgtgagaatcacctggattctgggatgctttgtgaaatgaaatgaagattcccgggtccataccctaccccctgcccccaacagccacagtctcttgggacagagcctagaaatcttgcctttgctaagcacctcggtagatttttatgcacagcaaaggttgagaaccactacctcttgttttgctgctgaaagtgataaaatgtgccaggaattttggaagtacttattaagccaatctgaacatcaaggagccatttaagtcagtaactcagaggaataagtagagtaaaaatgtcataaactctcaataaaagcaatcaatttaacaccaggagtaataaatgcataaaatgaagatgagttatctaatagagaaattatataaaccatgattataactctatatttgagttcccccttttccgtaatcagttaattttctaaaaaatcttcgtcacttaattctagcttgatcagatcccttcagtccgtaactccctgctcctcatcttagtttagcccttcttttttcttatgccacctttcctaaggaccagagaagtgaaatgataatatattggccacctacaatgttctagacatcatacatgtattttctctgctcttctgcataatcactgtgaggcaggcaatactcctccatttcattggggaggacattgaggttctgaactagtgggtcagttgtcctttttctgaatttgattacccagtagtataaagctttcttaggtaactcacctttatcacttgctgactgaattctgacagatgtcagtttctaattatagcctggacattcagatgtattcaggaccaagttgtcctcactctacctacaggcatgaatttctctcattgactaggttaggagcgccatatgtctgcagcctccctcagaatcccctgtgttctcacaccagggaactgagggttccctgggtccttccaggtagaagttcattgtacaatgaaacatcccttaaggaccatttcatctcttctttaggtgcatcacacatggttaaaacaaagtaataacagaacttagaatggaatcaaacagaatgaaacttacaccaagtacaattctcattacattaacccagagaagtgaaaagtagaagaatatttatttcaagccaatataatttccaagggctttgttgaaggctgaaatcttcgggaggaaagtagtgagaagaaaactgttcattcctctattttcccagtatataattgttttgatcattttctttcctttccagggactaaagacatgtggaaagcctactctgacatgaaagaagccaattacaaaaaattcagacaaatacttccatgcttgggggaactatgatgctgtacaaagggggcttggggctgtctgggctacagaagtgatcaggtaatgcacattcctgatgttgccaggaatgagtgagcagagcttgactgccttggacagtcaggagagaggtaagctccttgcagagaagttagaggctgcagcccctcctcctcttgccctctctctgcctgtgtgcttagtgcgagggtctgagtggatggtagaagtgagtgattcctcaccctccctctctgggtgctgttcatccagcctaggggtgcccagcctggctgagtggggcagtgcccaggcagggtcattgttttcacccctccttccttggccttcctgggcttctcccagagtcctcccttggaaagcagagaatgggaaggtgggctgttgctcactggcctggtgattaatctccttgcttgcctggactacagcgatgccagagagaacgtccagagactcacaggagaccatgcagaggattcgctggctggccaggctaccaacaaatggggccagagtggcaaagaccccaatcacttccgacctgctggcctgccagagaaatactgagcttccttttcaatctgctctcaggagacctggctgtgagcccctgagggcagggacatttgttgacctacagttactgaattctatatccctagtacttgatatagaacacataaaaatgcttaataaatgcttgtgaaatccagtttgttattggaatctggaagcagaatatgacagtcttcctgggatcatgggcctgtttagtaccatagggatgaccaataaac

SEQ ID NO: 6 - DNA, 193 bp, homo sapiens
gttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtctgggct

SEQ ID NO: 7 - protein, 64 aa, homo sapiens
VFCSLVLGVSSRSFFSFLGEAFDGARDMWRAYSDMREANYIGSDKYFHARGNYDAAKRGPGGLG

SEQ ID NO: 8 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactga

SEQ ID NO: 9 - protein, 122 aa, homo sapiens; identical to SEQ ID NO: 4

SEQ ID NO: 10 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccgtggtgcggaggactcgctggccgatcaggctgccaataaatggggcaggagtggcagagaccccaatcacttccgacctgctggcctgcctgagaaatactga

SEQ ID NO: 11 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 12 - DNA, 10001 bp, homo sapiens
gggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctggtaatccatagcccatggcctggctttacttctgtaaagtggcaggagaccttttgacagctgagccatttcttattttatttattttaataagagatggtaggaatgagcaatgatattagtacctggggactgttgttcttaaggagaaacaatcttagaatgattagtgataccccttgctttctcttttctttcattatactttttgtacacatatttttcccatttatttattggaatcttactgatttattataagtataagctttatgtctacacatgtataatcatttttccccaagtataagtctctttttcatggaggcacagcctagacctggttagccgccatctcccctcattgtatgcccaatatctattgtagtatctgctgcatagaaggcactcgatgcgtgaatggataatgactgatgatgaatcaataaataaatggacatgtcattgtaaaaaattctaaaaatctagaataacacaagctgttggcactacctagaaacacagatgtaaaacttcctaggttgtgtttcaccatgggaacatgtctttgaacaaaaatgggatcatattctattgcactctttcccttaagagatacttctccaggtcattaagtgctcttccacaatatcagtatatggcagaggcaaggtcataccaggtctgtctgaaaccagggcttggctcttaacttgcagccatactgcctccaagtctaggtggctgggttttaggatctgtaatgggaactcagtgtcacaacctctactgggaaggtattctggtgttgcataacaggactttctgttagagataaccatggcaaaatggaatagagacaaagttcaggtttctgctgccaggagctgagattgctgtgaccaatggcattctcccaaaccaaataatccaacctggaattaccataaaccactcctcatcttttcaaggggtgtccaagttcccagaaaagaacatttgttaagggatggaggcaaggaggtggagaagaaagagcactggccaaggtatcatgagtgtcctgggttctggtccttgaataagccatttatcttctctgcagcttctccatctgataggagtttggaggcagagttttttcttaatgagcaaaagacagtcgtgcctaggagatgtggtgtacatgttagaaagaagggactggctgtgactctataaaagatgaattcatacaaaaacaaattaccctttcccagggagaaagtttggatccagtaattagagatctcaaaaagtagaagacctgccctgtgaggcctgtggcctccaagtttgaatgctgtgtgtcagctttaaaaactagtttcttgctgataaatgtttcatattaagcatgtgttgagagtactccttgcctaccttcactagccactgtttccttcccctcctcccttgtcccttcattctctccagaactttctgctaacttccattctcttcaggacttcagcatggttgggagaagatcagaaaggcatcctcactgtttttattttagtccacttgacctttggggagtagttccactggctcataagtatcagccccccatagcacagcaccccacactgagcccggaagcaataaagaatcccaatctgctgtcactaaccagcacgctcaactgccatgccctttactcttctcatctccctgctttcacgtcacaccaactaatttctctatgagtcagcctcaactctcccaacactctgcccacccttcttctactaccttccagtgagctcctcgaaagaagggtctgcggtgaggatgcccctttatctctgcctatttccttcccattacaaaaacttgaaacctgcctttcccatgttgatttcactttattctcatctttacccatggggtatgcctcctgcaattcctcctagacaatagaatgagaaagaggggtcctcgtcctctttgctttccatgaccatttctccattcttcacctctgtgatgtgtcctctttgaagtccctgataaattcattaccaccttctctccagtcttactaatgttatctgcacaagtgatttccaaacaggaagattttcaaacactgattcctgaagatcacccccaactcgctgaactgagaccaagacctccaagattatggcttaggaatctgcattttttttttttttttgagacaagagtctcgctctgttgccaggctagagtgcaatggtggaatcatagctcattgtaacctcaaactcctgggctcaagtgatcttcctgcctcagcctcccaagtagtgaggacaacaggagtgtgccaccatgcccagctaattgttaattttttgtagaaatggagtctcactatgttgctcgggctggtctcaaactcctgaccttaacccatcctccgcctccgcccccaaaagtgttgggattacaggtgtgagccaccgtgcccagcctagaaatacccactagaagcttctgtgtagacaatctgcttagtgatgtttggagacaaagtacctctttattgtattcattgacaaaactctccagtcctctcccatcttcatggaaaattttcacagttcatttacggccctctttccaacacattcactgccaatactcttattgacaataactgtattgttgaaccttccagtatcctgcattcccggatcaaggccccctcaaagccctgatatgcaaatatctgggaaaagaatgttccagaggaaaggaacagctaatccgaggcccctagggtaagatgtgcctgggggtttggagaccagtgtggccagagcaaaatgagcaggaggagagaattggatgatgaggtacgagaggaaggagttaggacagtttgagtaaagtttgaaaaccattataagggctttgacttcaactatgagtggaagtggaatcctccggagagttttgaatggagagtgatagaagttgtcttgtgttgtaacagtctggctgctatactgaaaagagactagttggcggcaaagggggaaatgtggaagccagttaagaagccatcataacccagaaggtgatgcctaataacatctctctgggagcagcggagagatgataagggtttgccttctgaatatgttttttgacaattaatgtaaacatttcaagtaggctgagattttattgcatattaacaatgtccatgttcactcgcggcagccgcccccttctgcgcggtcatgccgagccagcacctgggcctggaactgggccgcagcccccagcttcacccaccacctccctaccatggacccctgcaaagtgaacgagcttcgggcctttgtgaaaatgtgtaagcaggatccgagcgttctgcacaccgaggaaatgcgcttcctgagagagtgggtggagagcatgggaggtaaagtaccacctgctactcagaaggctaaatcagaagaaaataccaaggaagaaaaacctgatagtaagaaggtggaggaagacttaaaggcagacgaaccatcaactgaggaaagtgatctagaaattgataaagaaggtgtgattgaaccagacactgatgctcctcaagaaatgggagatgaaaatgtggagataacggaggagatgatggatcaggcaaatgataaaaaagtggctgctattgaagtcctaaatgatggtgaactccagaaagccattgacttattcacagatgccatcaagctgaatcctcgcttggccattttgtatgcaaagagggccagtgtcttcgtcaaattacagaagccaaatgctgccatccaagactgtgacagagccattgaaataaatcctgattcagctcagccttacaagtggcgggggaaagcacacagacttctaggccactgggaagaagcagcccatgatcttgcctttgcctgtaaattggattatgatgaagatgctagtgcaatgctgaaagaagttcaacctagggcacagaaaattgcagaacattggagaaagtatgagcgaaaacatgaagagcgagagatcaaagaaagaatagaacgagttaagaaggctcaagaagagcaggagagagcccagagggaggaagaagccagacgacagtcaggagctcactatggcccttttccaggtggctttcctggtggaatgcctggtaattttcccggaggaatgcctggaatgggaggggacatgcctggaatggccggaatgcctggactcaatgaaattcttagtgatccagaggctcttgcagccatgcaggatccagaagttatggtggccttccaggatgtggctcagaacccagcaaatatgtcaaaataccagagcaacccaaaggttatgaatctcatcagtaaattgtcagccaaatttggaggtcaagcataatgcccttctgataaataaagccctgctgaaggaaaagcaacctagatcaccttatggatgtcgcaataatacaaaccaacgtacctctgaccttctcatcaagagagctggggtgctttgaagataatccctacccctctcccccaaatgcagctgaagcattttacagtggtttgccattagggtattcattcagataatgttttcctactaggaattacaaactttaaacactttttaaatcttcaaatatttaaaacaaatttaaagggtctgttaattcttatatttttctttactaatcattgtggatttttccttaaattattgggcagggaatatacttatttatggaagattactgctctaatttgagtgaaataaaagttattagtgcgaggcaaacataaaaaaaaaaagtccatgttcatctctaaatgacatcattgttccaaagcttttccattcttcttaaccttccacctgtcaatctataggagatgacttctcctacttcactcatgcattgactccttcaatcaataaaagtgactaagaacctgctacaggtgaggtgctgtgtttggtgttaaagtgacaacagttatctgtcaataagcctgacaaggttcctatccctgtgttttgtgcactctgggtcaaactcagaaatgcaaacaggtggagagcgatgagttctatgactggtaaagaaaagggcctgctggtttccctcaggatctctgtccttcatctcaaaatgcatcttccttgttatcgttcctctccttcctgtctcagaggaagacctgctcctgctacactctgggcaaccttgtccccgtggccctgtggccccttggttgttgaagtctatgttatgccctatcttttaccctcagtcactctctctgttaacattctccctgtgccctgtaaccctccctcatctttaaataaatcctcctcctttgaccttcgcatgtattcagtcatgcaactcaacaagcatttattgcacagtgatattcaatttgccacttgctaaaagtctgaaccttggcagctgaatgtgatcagaaaaaaagcacgactgctatgactagtctcactttaaattcatggtcgttgaccaagagctaccatacaatccactacctttctcaagttcagtcacattcttcctttcctagatgtctgctttctacttctcttctcttctgaaacttcccacaactcctcgttcattctcttctcagttgacaactttgcttcctatttcactgaaaaatagaagcaatcagatatgaacttctggctgggcatggtagctcatgcctataatctcagcactttgggaggccaaggcaggaggactgcaggttaggaatttgagaccagcctgggcaacatggtgaaactcccactgtactaaaaattttaaaaattactcaaacatattggcaaacaactgcagtcccagctacttgggaggttgagatgcaaggatcacttaaacctgggaggctgaggctgcagtgagccatgattgcaccactgcactccagctcaggcaacagagcaagaccctgtcttgagaggagaggagaagagaggaggggaggggagggcaggggaggggaggggaggggaagggagaggggaggggagaggggaggagagaggggaggggaggggaggggaggggaggggaggagaggaggatcaggtgaggagtatgccaaggagtgtttttaagacttactgttttctctttcccaacaagattgtcatttcctttaaaaagtagttatcctgaggcctatattcatagcattctgaaagaaagaaaagaaaagaggaaagaaagagagaggaaggaaggaaggagaaagagagaggaaggaaggagaaagagagaggaaggaagggaggaagagaagaagggaggaagaaaagaaggaaggaaggagggagggagggaagggagggagggaaagaggaagaaaggagggaaagaaggaaggaagagagagaggaaggaaggaggaagagagaagaaggaaggaggaagacagagagggagtaaggaaggaaggaaggagaaagagagaggaaggaagaaatgaaggaaggaaggaaagaaagaaaaaataaaagagtgaaaacggactggagaagaagaaaccacagttgctgctatatccaccagcctctctgcatgtcctggcctcagccctgctgggctctggtactgaccacttccttccttcctaatttcctaattgactaggccagctgagcagggcttttctgtgctgaggaggtaaatctctggatatctagactgaggggtggaaggagccttccagggcacacatgagacatggcaggggtaggctgctagttttattttgttttcttttagacacagggtcttgctctgttaaccaggctggagtgcagtggcgtgattatagctcactgcagccttgacctcctgggtctcccacaatccttccgcttcagcctcttgagtagctgggactgcaggtgcacactaccacacccggtccatttatttttatatttcgtagagacaagatcttacagttttgcacagagtgatcttaaactcttgaccccaagtgatcctcctgccttggcctccaaaagcattgggattataggagtgagccactgtgctggacctagtctgtcagctttgaagctttagatatgaactcagagggacttcatttcagaggcatctgccatgtggcccagcagagcccatcctgaggaaatgactggtagagtcaggagctggcttcaaagctgccctcacttcacaccttccagcagcccaggtgccgccatcacggggctcccactctcaactccgcagcctcagccccctcaatgctgaggagcagagctggtctcctgccctgacagctgccaggcacatcttgttccctcaggttgcacaactgggataaatgacccgggatgaagaaaccactggcatccaggaacttgtcttagaccgttttgtaggggaaatgacctgcagggactttccccagggaccacatccagcttttcttcgctcccaagaaaccagcagggaaggctcagtataaatagcagccaccgctccctggcaggc

SEQ ID NO: 13 - DNA, 10001 bp, homo sapiens
gtctgccagggagaggtggctgctatttatagtgagccttgctggtctcttgggagggaagaaaagctggatgtggtccctggggaaagtccctgcaggtcatttcccctacaaactggtctaagacaagttcctggatgccggtggtttcttcatcccgggtcatttatcccagttgtgtaacctatgggaacaagagaggtttgctgtgccttggcaatggacagggtgctagatcagctctgctcctcagcattgggggaagtgcagctgcagagatgccagtgggagccccgtgatggcggcacctgggctgctggaaggtgtggagtgagggcagctcttcagccagctcctgactataccggtcatttcctcaggatgggccctgctgggccacatggcagatgaccctgactgaaatccctgtgagttcatgtctaaagctttaagctttaaaacggacagcctacccctgccacatctcatgtgtgccctggaagcctccttccacccctctggatgtcctgatatttctcagcacagaaaatctctgctccgctggcttagccaatttggaaatgctttttctaagttggctcctgagccaaggacaatgtagagagggggactttctgctgccccagcctagtcctggagccccaccttgggagaatgagagtgtggtgcgttaaataggcagcccagctggggacgtgcccagcatccaggcagggaagggtgggagagctcttggtctgctgtattatcacggaggggtgcagggggcatgcagatcactctctcatgagaacatcaacagggtcagattagctctgcagaggcttatggaggagcatggtggccagagatgggtcagtaccagagcccaggggggctgaggccaggacatgcagagaggctggtggacatagcagcaactctggtttcttcttctccagtccatgttcataccctgagggctaggcatttgtaataacaaacaaacaagcaatttagaaatgggccaggcatggtggcatgtgcctatagtcccagctacttgggaggccaaggcaggaggcctgcttgaacccagaaatttgaggccagcctgggcaacacagcaagattatcttaaaaaattttttttaatctctgagaaatgggtagggccaggaagtaaaggatggccaaatactccataagcagcaaatgcgtggctccaatgtgaacaatgatattatagactctgttctgagacctatgcattgacacctccacctcccccactacatcttgccaccttaaaaccactgagagtggtacctgctggaatgggtccacacacacagtcacacatattttaggcagggtagttgacatccccagggaaaaagagctcacagagagaggctgaatgtttccaactgggtagcagtaatagtacatcatgctgtacatggtacagcacagatcaggtgaaaataatagcacatcgtgattaaccagggcttattccagggagtcaagaagagtttcatatcagaaaaatctatctttgtaattcactataccagtaatcaaagaaaaggattgtacatttattttactagatgcagaaaatgaatttcataattgtcaacatctactgatgataaggaaaatgtataacaaaataaagagaccatttctgacttgagaaaggataaataccaatatgttatagcaacagttctcaaactgttttccagggaaccctaagaatccctccttagggaggctttgatctcaaaattatttttagaatagtgctaacacactattttcatgtttcagtctcattttctcatgagtacacacaatatgacaagttagttgatatgagtgtggatttccacatggtaactgacttttcagaagctaccacttgttgagtttggtataatatagaatagccacaattatctaaaaataccattaaaatacactcccccatttcaactatatatctgtgtgaggctgaattttcttcatatactccaacctaaataacatattaaaacaggttggatgatgaatcagataggaaaatccagctatgaaaaaaaaatcagacatgaaaaattttcaaaagggtaaaaccatagtactcttcttactttttttcttttggaagatggttatttttcataaaaatatattatttatgttaacatatagaagatggataattttttgaagaattgataaatgtttaaattttttctttctattatggtaaatactgatgaatagagtccccataaataaaagttctttggggtattcaataatttttaatagtgtaatgggatcctgagaccaaaaggtttgagaatcattgctctacagcaaacattatgtgtaattaagacacttcaggtgcattctcaagaagaccaataaagaggccacaatggcaggcgtggtggctcacacttgtaatccaagaacttagagaggacgaggcaggtggatcactggaggtcaggaattctcaaccagcctggccaacatggtgaaaccctgtctctactaaaagtacaaaaattagtcgggtgtagtggcaggtacctgtaatcccaagtacttggggggttgaggcaggagaatcacttgaagccgggaggtggaggctgcagtgagccgagatcgtgccactgcactccagcctgggcaacggagtgagacttcatcatggaaaaaaaaacaaagaggccaggatgtctggttgttactgccactgtttcacatatccctgaaggacctgcccaatgctaaagaaacacaaggaaggtaagaggtgaaagagaagaaatgaaactatcattgtttgaagatgacaccatcttttacatagaaaacctgttagaatcaaatggcaagctattagaactactaagagaattcagtgaggctgctgtattcatggcaaaattttaacaattgatagcatttctctgcaacattccttaatagttataaaatacagcacaaagtagtaccaaaaatattaactatctaggaaataacctcttacagagaaaatttagtctgttaaaggataaacagtggcaatgtacgtcatgtccacagagattatattttagcttagcaaagataccaattctcccaaatttatttataaattaaatgcaatgtgaatcaaaatttcccactggaatttttatcaggaaggcaacaaattctttctttctttctttctttctttctttatttatttatttatttatttatttatttatttccttccttccttccttccttccttccttcctttctttctttctttctttctttctttctttctttctctctctctttctctctccccccctctctctctctctgtctctctctctctctctttctttctttctttctttctttctttttaagacaaagtctggctctgtcacccaggctgcagtgcagtgatacaatctcagctcactgaaacctcaacctctccggcatcaggtgaacctcccacctcagccccccgagtagctgggactacaggtgcacaccactgggcctagataactttttgtatttattgtaaataaacacaaaaaataaatattttgctcaggttggtctggaactcctgggctcaagcaatccgcctgccttggcctcccaaagtgctagaattacagttgtgagccaccacacccagccaataaattaattctttatgatgaataagttatctatgaaaattaagtcagctgggtgcggtggctcacgcctgtaatcccagcactttgccgggctgaagcaggtggatcacctgaggttgggagttcaagaccagccggaccaacatagagaaaacccgtctctactaaaaatgcaaaattagctgggtgtggtggcatatgcctgtaatcccagatacttaggaggctgaggcaggagaattgcttgaacccgggcggtggaggttgcggtgagccaagattgcaccattgcactccagcctgggccacaagagcgaaactccatctcaaaaaaaaaaaaagagaagttaagtcaatgaaaagttaagtcaattaaaaaagtaagagctgtagtgtttagatatatacacacacacatatatatatatttatctttatatatgtatatatatcttttcctttttttgagaccgagtctgtttttgttgcccaggctggaatgcagtggcgcgatctctgcttactgcaacctctgcctcccaggttcaagcgattctcgtgcctcagcctcccgagtagctgggattacaggtgcctgcccccatgcccggctaatttttgcatttttagtagagacggggtttcaccatgttggccaggctggtctcaaactcctgacctcaggtgatccaccggcctcagcctcccaaagtgctgggattacaggtgtgagccaccgcgcccagccatatattttgcttttcatctgcagctcctggatcctaactccttgttatattgttgggcactttaggcctcagtaaacagaatctctgtctatgaccttctcctgtccttcttccacctgcccaaagcaggactctaatttgattgtgggtcaaaagactctcattccagaaagggccttgcctcataccctagaggaaggaatgctgcacagaaacgccaagtctgaacagacaagccttgctgggtttataccatatgctttttgtccaatcacatttcttcatggttgccaatcatgcctatgtaatgaagcctccataagaacccagaaggacagggttcagagagtttccacatagctgaacactatctggagagtgaacacttcctagagagtggcacacccagagagatcatgaaagctccacgcccctttccccttacctcgccctccacatctcttcatctgtatctttcataatatcctttataaataaaccagcaaatgtgtttccctgagttatgtgagtcactctagcaaattaatcgaacccaaagagggggtcatgggaaccccaacttgaagccagtcagtcagaagttccagaggcccagacttgcaactggggagaaagagggggaggtcttggggactgagcccccaacctgtgggatctgacactgtctccaggtaggtagtgttggaactgcattggaggacactcctggtgtctgctgcttggtgtgtggggggaaaaacccacacctttggttacggaggtcttctgtgttgacgatcattgctgtttgagggcagagggaatacacggtttgagagagtttttccctgacatgagcgaacaggggacatgtactggtctctgagatgggggatcatgggatctgccacaagtggggagaccactgtgacccctgccacagtctttggggcagagggtgtctcgggggcagaagaagcgagagttgtttgcagtagcagttatgtccaaagtgggcgccaggaaagtagggctgcccagctttgaagagcctccttactcccagcctgaatgaaaccatttcctgtaaagcgctaagcataaagtttgccaatggtgatccacggagaagtgagtgtaccccaccccgccatcccacagggaatgtcggagtgatgttgatctgcacctagggaaggaatggttcatgagatgtggtggagatgctgagggcccgtggacatcagatcctaccctacctgtgccaggacaagccatgcgcatgtgcttcagaccaccaggcaacaggagtgttgcatgaggtgtgaagcaggcacctgggaaagaggagtgtgaacagcagatgggacacactgggggcagtcataggaatgaaatgtcccaggatggatgcaggcaggttatggaggacttagtgaggactgctctcctggtgggaattgtggagtgggagactggatggagactggaggtgttttaagtagggaagccaacttgcaagggtgaccagggaaactatgtcggccaagggtgagacatgcactggcaagactctcagacagcctggcttatctaagcagaatgcttgagccatgccaacggtgcctcgcaagttgtattaatcatgtcctttcattttgtgtttttggtgcttggcatctgggcccttgctgaccctaagggaccatttctctcagagctagtcaagtcctagacacagtaaatgactctcctgggagcatgccttccatgtgcagaccaaccaatcaagagtccacactcccacccacctcctttatcgagctctcacatcctggggcaccatccacctgccctaatcactcaaggaccacgtcccaaacaactagggacagcctccatgcccctgcacccattgaaattattcatgctagccaatcctaaacctgtgtatgctgccacaccattccttcctgcagaaacacagtaaggactcttcctacacctcccctacttcctctgctccctgacttacccacttacttcctggtgcagtcccctgtggcatagttcactctcttcttttgggaactgtgaggctatcttctcaatggcagtcatctcctgagctgttggccttgccatacctaactaataataaaatctatattctaaggtaaaaacaaaacagatagggtctcactctgttgcccaggctggagtacagtggtgtgatcatgactcactgcagcctcaaactcctgggctcaagcagttctctcatctcaacctcccgagtagctgggactacaggcacacaccaccatgcctggctagttttcttattttttttgtagatacagggtcttgttatgttgccaaggctggtcttgaactcctgggctcaagtgatcctcctgccttggcctcccaaactgctgcaattacaggcatgagccaccatgcccagatcagaaatcttactaaaaatatttcaaggagaagagaaagccaaagatgttgaatatatatatatgtgtgtgtgtgtgtgtgtatatatatgtatatatgtgtatatatgtgtgtatatatatatgtatatatgtatatatatatgtatatatgtatatatatatgtatattggggcaggcgtggtggctcatgcctgtggtcctaactacttgagagtctgaggtgggaggattgcttgagcctgggagatcgaggctgctgtgagctgagactacaccactgcactccagcttgggtgacagagtgagaccctgtctccaaaaaaacaaaaagaaaaagaaaaaaagatggaaaaagacatgaaaaaacaacaacagaaatacccacacatcatcaatgggagggaagcatcttgaggcagcaaagcgggagtgctagtagagaggcagatagggcgttggacctgaggcattaaggaaagtcaggatttggagcttacaagtctctcattggagatgggatggggttggaatgaatgtctgagcaaacacaaagcatttccttccctaatgactccccaccagtctaaagaatcccacattaggtcgaacacggtggctcacgcctgtaatcccagcactttgggaggccaaggcgggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctg